(19) United States
(12) Patent Application Publication
(10) Pub. No.: US 2010/0311698 A1
(43) Pub. Date: Dec. 9, 2010

(54) AQUEOUS BASED PHARMACEUTICAL FORMULATIONS OF WATER-SOLUABLE PRODRUGS OF PROPOFOL

(75) Inventors: Kanaiyalal R. Patel, Union, NJ (US); Stephen Dordunoo, Baltimore, MD (US); David Keller, Baltimore, MD (US)

Correspondence Address: BANNER & WITCOFF, LTD., 1100 13th STREET, N.W., SUITE 1200, WASHINGTON, DC, 20005-4051, US

(73) Assignee: EISAI CORPORATION OF NORTH AMERICA, Woodcliff Lake, NJ (US)

(21) Appl. No.: 12/443,583

(22) PCT Filed: Sep. 20, 2007
(86) PCT No.: PCT/US2007/020351

§ 371 Date: Aug. 11, 2010

Related U.S. Application Data
(60) Provisional application No. 60/828,307, filed on Oct. 5, 2006.

(51) Int. Cl. A61K 31/661; A61P 23/00; A61J 1/05

(52) U.S. Cl. 514/130; 514/76; 604/403

(57) ABSTRACT

The present invention is directed to aqueous based formulations of water-soluble prodrugs of propofol. The formulations comprise in aqueous medium an effective amount of the water-soluble prodrug of propofol in the absence of an antioxidant. The formulations are particularly useful as intravenous injections. The formulations preferably are buffered to a pH suitable for minimizing degradation of the prodrug during storage. The formulations can be prepared without the use of harmful co-solvents or surfactants and are stable at room temperature over extended periods of time.

### BACKGROUND OF THE INVENTION

The use of injectable anesthetic agents generally, and of propofol specifically, in the induction and maintenance of general anesthesia has gained widespread acceptance in anesthetic care over the last 15 years. Intravenous anesthesia with propofol has been described to have several advantages over preexisting methods, such as more readily tolerated induction, since patients need have no fear of masks, suffocation, or the overpowering smell of volatile anesthetics; rapid and predictable recovery; readily adjustable depth of anesthesia by adjusting the IV dose of propofol; a lower incidence of adverse reactions as compared to inhalation anesthetics; and decreased dysphoria, nausea, and vomiting upon recovery from anesthesia [Padfield N L, Introduction, history and development. In: Padfield N L (Ed.) Ed., Total Intravenous Anesthesia. Butterworth Heinemann, Oxford 2000].

In addition to its sedative and anesthetic effects, propofol has a range of other biological and medical applications. For example, it has been reported to be an anti-emetic [McCollum J S C et al., Anesthesia 43 (1988) 239], an anti-epileptic [Chilvers C R, Laurie P S, Anesthesia 45 (1990) 995], and an anti-pruritic [Borgeat et al., Anesthesiology 76 (1992) 510]. Anti-emetic and anti-pruritic effects are typically observed at subhypnotic doses, i.e., at doses that achieve propofol plasma concentrations lower than those required for sedation or anesthesia. Antiepileptic activity, on the other hand, is observed over a wider range of plasma concentrations [Borgeat et al., Anesthesiology 80 (1994) 642]. Short-term intravenous administration of subanesthetic doses of propofol has also been reported to be remarkably effective in the treatment of intractable migraine and nonmigrainous headache [Krusz J C, et al., Headache, 40 (2000) 224-230]. It has further been speculated that propofol may be useful as an anxiolytic [Kurt et al., Pol. J. Pharmacol. 55 (2003) 973-7], neuroprotectant [Velly et al., Anesthesiology 99 (2003) 368-75], muscle relaxant [O'Shea et al., J. Neurosci. 24 (2004) 2322-7] and, due to its antioxidant properties in biological systems, may further be useful in the treatment of inflammatory conditions, especially inflammatory conditions with a respiratory component, and in the treatment of neuronal damage related to neurodegeneration or trauma. Such conditions are believed to be associated with the generation of reactive oxygen species and therefore amenable to treatment with antioxidants. See, e.g., U.S. Pat. No. 6,254,853 to Hendler et al.

Propofol typically is formulated for clinical use as a oil-in-water emulsion. The formulation has a limited shelf-life and has been shown to be sensitive to bacterial or fungal contamination, which has led to instances of postsurgical infections [Bennett S N et al., N Engl J Med 333 (1995) 147]. Due to the dense, white color of the formulation, bacterial or fungal contamination cannot be detected by visual inspection of the vial in the first instance.

Not only is propofol poorly water soluble, but it also causes pain at the injection site, which must often be alleviated by using a local anesthetic [Dolin S J, Drugs and pharmacology. In: N. Padfield, Ed., Total Intravenous Anesthesia. Butterworth Heinemann, Oxford 2000]. Due to its formulation in a lipid emulsion, its intravenous administration is also associated with undesirable hypertriglyceridemia in patients, especially in patients receiving prolonged infusions [Fulton B and Sorkin E M, Drugs 50 (1995) 636]. Its formulation as a lipid emulsion further makes it difficult to co-administer other IV drugs. Any physical changes to the formulation, such as a change in lipid droplet size, can lead to changes in the pharmacological properties of the drug and cause side effects, such as lung embolisms.

It has further been reported that the use of propofol in anesthesia induction is associated with a significant incidence of apnea, which appears to be dependent on dose, rate of injection, and premedication [Reyes, J G, Glass, P S A, Lubarsky D A, Nonbarbiturate intravenous anesthetics. In: R. D. Miller et al., Eds, Anesthesia. 5^{th} Ed. Churchill Livingstone, Philadelphia, 2000]. Respiratory consequences of administering anesthetic induction doses of propofol, including a reduction in tidal volume and apnea, occur in up to 83% of patients [Bryson et al., Drugs 50 (1995) at 520]. Induction doses of propofol are also known to have a marked hypotensive effect, which is dose- and plasma concentration-dependent [Reyes et al., supra]. The hypotension associated with peak plasma levels after rapid bolus injection of propofol sometimes requires the use of controlled infusion pumps or the breaking-up of the induction bolus dose into several smaller incremental doses. Further, the short duration of unconsciousness caused by bolus induction doses renders propofol suitable for only brief medical procedures. For all the above reasons, propofol for induction and/or maintenance of anesthesia must normally be administered in an in-patient setting under the supervision of an anesthesiologist, and is often considered inappropriate for use by non-anesthesiologists in an ambulatory or day case setting.

In addition to its use in induction and maintenance of anesthesia, propofol has been used successfully as a sedative to accompany either local or regional anesthesia in conscious patients. Its sedative properties have also been exploited in diagnostic procedures that have an unsettling effect on conscious patients, such as colonoscopy or imaging procedures. Propofol has also been used as a sedative in children undergoing diagnostic imaging procedures or radiotherapy. A recent development is that of patient-controlled sedation with propofol. This technique is preferred by patients and is as effective as anesthesiologist-administered sedation.

Compared with the widely used sedative midazolam or other such agents, propofol provided similar or better sedative effects when the quality of sedation and/or the amount of time that patients were at adequate levels of sedation were measured [see Fulton B and Sorkin E M, Drugs 50 (1995) 636]. The faster recovery and similar or less amnesia associated with propofol makes it an attractive alternative to other sedatives, particularly for patients requiring only short sedation. However, because of the potential for hyperlipidemia associated with the current propofol formulation, and the development of tolerance to its sedative effects, the usefulness of propofol for patients requiring longer sedation is less well established.

Due to its very low oral bioavailability, propofol in its commercially available formulations is generally recognized as not suitable for other than parenteral administration, and generally must be injected or infused intravenously. While propofol is administered intravenously in a clinical setting, it has been suggested that it could be delivered for certain indications via other non-oral routes, such as via inhalation using a nebulizer, transmucosally through the epithelia of the upper alimentary tract, or rectally in the form of a suppository [see, e.g. Cozanitis, D. A., et al., Acta Anaesthesiol. Scand. 35 (1991) 575-7; see also U.S. Pat. Nos. 5,496,537 and 5,288,597]. However, the poor bioavailability of propofol when administered by any other than the intravenous route has hampered the development of such treatments.

The development of water soluble and stable prodrugs of propofol, which is described in U.S. Pat. No. 6,204,257 to Stella et al., has made it possible to address these heretofore unmet needs, and to explore the pharmaceutical advantages of an orally bioavailable aqueous propofol-prodrug as a therapeutic agent. The prodrugs differ from propofol in that the 1-hydroxy-group of propofol is replaced with a phosphonooxymethyl ether group:

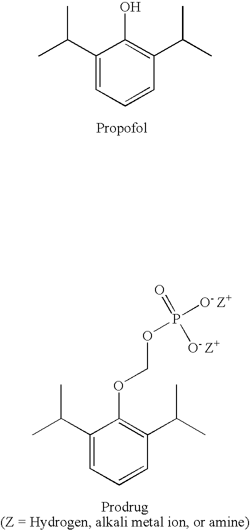

While the present invention is not bound by any theory, the prodrug is believed to undergo hydrolysis by endothelial cell surface alkaline phosphatases to release propofol.

Stella reports that the prodrug has good stability at pH levels suitable for making pharmaceutical formulations, and quickly breaks down in vivo under physiological conditions when administered intravenously. The prodrugs possess excellent properties for oral administration and a favorable pharmacological profile as orally bioavailable therapeutics for sedation and anesthetic care, and for the treatment of conditions such as migraine, epilepsy, pruritus, anxiety, insomnia, nausea, and other medical conditions.

In commonly-owned WO 2003/057153 A2, aqueous formulations of the above-described prodrug are prepared with an antioxidant. These antioxidant-containing formulations provide excellent stability, particularly when packaged in substantially oxygen-impermeable containers, such as glass vials. However, antioxidants can be consumed when formulations are packaged in containers that are more permeable to oxygen, especially many types of plastic containers such as blow-filled seal (BFS) vials. It would be desirable to develop an aqueous formulation that exhibits stability over extended periods of time, while avoiding the need for packaging in substantially oxygen-impermeable containers.

### BRIEF SUMMARY OF THE INVENTION

The present invention is directed to aqueous-based pharmaceutical formulations of water-soluble prodrugs of propofol. The pharmaceutical formulation comprises in an aqueous medium a therapeutically effective amount of a compound represented by Formula I:

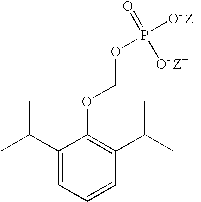

wherein each Z is independently selected from the group consisting of hydrogen, alkali metal ion, and amine. According to one aspect, the aqueous formulation is substantially free of antioxidant.

According to another aspect, the formulation includes a plurality, preferably two, buffers. The buffers can be selected to maintain pH within a prescribed range so that the formulation is stable for extended periods of time by minimizing degradation of the prodrug. In one embodiment, a combination of 2-amino-2-hydroxymethyl-1,3-propanediol (TRIS) and sodium bicarbonate is used as a buffer system.

In another aspect, the formulation is buffered in a pH range of about 8 to about 12, preferably from about 9 to about 10. It has been found that degradation of the prodrug is pH dependent. When pH is maintained within this range, the formulations exhibit stability over extended periods of time, without the need for an antioxidant.

The aqueous formulations of the present invention are particularly useful as intravenous injections. The formulations are stable for extended periods of time, and are suitable for packaging in plastic containers such as blow-filled seal (BFS) vials.

### DETAILED DESCRIPTION OF THE INVENTION

The pharmaceutical formulations of the present invention comprise in aqueous medium a therapeutically effective amount of a water-soluble prodrug represented by Formula I:

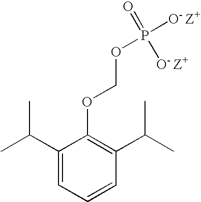

wherein each Z is independently selected from the group consisting of hydrogen, alkali metal ion, and amine. In one aspect, the formulation is substantially free of antioxidant. The aqueous-based formulations may also contain other components, such as a tonicity modifier and/or buffer(s).

Methods for synthesizing the derivatives of Formula I are described in U.S. Pat. No. 6,204,257 B1, the disclosure of which hereby is incorporated by reference in its entirety. A representative example of a compound of Formula I is O-phosphonooxymethyl-propofol, the structure of which is illustrated below:

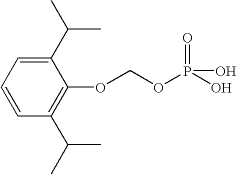

The relative amount of the prodrug in the formulation can vary over a wide range depending on a variety of factors including but not limited to the identity of the prodrug, the bioactivity of the parent drug for a particular disorder being treated, and the intended mode of administration. The relative amount of the prodrug in the formulation most often ranges from about 0.5 to about 20% (w/v), more usually from about 1 to about 10%.

Any pharmaceutically acceptable aqueous medium, such as water of sufficiently high purity, may be used in the formulations.

It has been found that degradation of the prodrug is pH dependent. In particular, under conditions of pH<8, the prodrug undergoes oxidative degradation into poorly water-soluble compounds. The prodrug is believed to be converted to propofol (DIP) by aqueous hydrolysis (or by enzymatic processes in the blood). DIP in turn is converted to the related substances quinone and hydroquinone by an oxidative process. All three of DIP, quinone, and hydroquinone are poorly water-soluble. It is desirable to minimize the formation or presence of poorly water-soluble compounds in the formulations because even at low concentrations, these compounds impart a yellow color to the solution. Over time, the solution becomes hazy, and eventually particles form.

The pH of the formulation preferably is maintained to provide long-term stability of the formulation at room temperature. According to one aspect, the formulation is buffered in a pH range of about 8 to about 12, often from about 9 to about 10 or from about 8.6 to about 9.5. These pH ranges minimize hydrolysis of the prodrug while, at the same time, are suitable for intravenous administration. The solution may be buffered using any buffer effective over this pH range, e.g., carbonate, phosphate, borate, or glycine. The amount of buffer most often ranges from about 2 to about 50 mmol, more usually from about 5 to about 25 mmol.

In one embodiment, a combination of tromethamine (2-amino-2-hydroxymethyl-1,3-propanediol), commonly referred to as TRIS, and sodium bicarbonate is used as a buffer system. It was found that when 2-amino-2-hydroxymethyl-1,3-propanediol is used alone as a buffer, the pH of the formulation tends to decrease over time. When sodium bicarbonate is used alone as a buffer, the pH tends to increase over time. When 2-amino-2-hydroxymethyl-1,3-propanediol and sodium bicarbonate are used together, pH remains more stable over time.

Particularly for formulations intended for parenteral administration, it is preferable to make up solutions such that the tonicity, i.e., osmolality, is essentially the same as normal physiological fluids in order to prevent post-administration swelling or rapid absorption of the composition because of differential ion concentrations between the composition and physiological fluids. If needed, a tonicity modifier can be added. When used, the amount of tonicity modifier used most often ranges from about 0.1 to about 1% (w/v). Non-limiting examples of suitable tonicity modifiers include sodium chloride, glycerin, boric acid, calcium chloride, dextrose, and potassium chloride.

Other components may be present in the formulation. For example, in the case of a multi-dose vial, a preservative may be included, such as benzyl alcohol. The formulation also may contain co-solvents such as polyethylene glycol (PEG 200, PEG 400), propylene glycol, and/or ethanol. Concentrations of the co-solvents can vary over a wide range, most often from 0 to about 20%. Although the formulations can be prepared without an antioxidant as described herein, antioxidants can be used, for example when the formulation is packaged with materials having lower oxygen permeability so as to avoid antioxidant loss. Non-limiting examples of antioxidants include monothioglycerol (MTG), glutathione, citric acid, ascorbic acid, sodium metabisulfite, and sodium sulfite. EDTA, a metal chelator, provides protection from catalytic oxidation of phenols.

The formulations may be administered via any suitable route of administration. Formulations for intravenous injection may be packaged, for example, in a glass vial, in a pre-filled syringe, or in an ampoule. Because the formulations do not require an antioxidant, the formulations need not be packaged in oxygen-impermeable containers such as glass vials, but alternatively can be packaged in a variety of types of plastic containers such as blow-filled seal (BFS) vials. The formulations may be administered with standard IV diluent solutions, e.g., D5W, normal saline, or Lactated Ringer's solution.

When packaging the aqueous-based formulation in plastic containers, other steps may be taken in addition to or instead of providing the formulation substantially free of antioxidant as described herein. For example, the formulations can be packaged in blow-filled seal vials by vacuum sealing in pouches made from an oxygen- and carbon dioxide barrier material, such as aluminum foil, and/or by adding oxygen- and carbon dioxide scavengers to a packaging material. Alternatively, blow-filled seal containers can be constructed using materials (e.g., multilayer) which have lower permeability to oxygen and carbon dioxide. As illustrated in Table I below, each of these techniques is effective for stabilizing pH and avoiding loss of antioxidant.

TABLE I
Stability of Aqueous Formulations in BFS Vials

| | | | Vacuum | Pouched with | |
|---|---|---|---|---|---|
| | | | seal | O₂ + CO₂ | Coextruded |
| | Time | Unpouched | pouched | scavenger | multi-layer |
| pH | 0 | 8.4 | 8.3 | 8.3 | 8.5 |
| pH | 2 weeks | 8.3 | 8.3 | 8.3 | 8.5 |
| pH | 4 weeks | 8.2 | 8.4 | 8.3 | 8.5 |
| pH | 12 weeks | 8.0 | 8.4 | 8.6 | 8.1, 8.7 |
| MTG | 0 | 0.16% | 0.19% | 0.21% | 0.20% |
| MTG | 4 weeks | ND | 0.14% | 0.19% | 0.15% |
| MTG | 12 weeks | ND | 0.11% | 0.16% | ND, 0.12% |

ND = Not detected

Suitable dosages can be ascertained depending on such factors as the identity of the prodrug and the type of the disorder being treated. Dosages may be, for example, in the range of about 0.1 to about 100 mg/kg of body weight, or about 5 to 500 mg/ml. As will be apparent to persons skilled in the art, many factors that modify the action of the drug will be taken into account in determining the dosage including the age, sex, diet and physical conditions of the patient.

For administering the propofol prodrug, an anesthesiologist skilled in the art of anesthesia will be able to ascertain, without undue experimentation, an appropriate treatment protocol for administering a formulation of the present invention. The dosage, mode and schedule of administration are not particularly restricted, and will vary with the particular indication. The formulation may be administered parenterally. The dosage may be, for example, in the range of 0.5 to 10 mg/kg administered according to procedures for induction of general anesthesia or maintenance of general anesthesia. Alternatively, the formulation may be administered by parenteral infusion, the dosage may be, for example, in the range of 2 μg/kg/min to 800 μg/kg/min administered according to procedures for maintenance of general anesthesia, initiation and maintenance of MAC sedation or initiation and maintenance of ICU sedation.

### EXAMPLES

The following examples are provided to illustrate the present invention and should not be construed to limit the scope of the present invention as described herein.

### Example 1

This example illustrates that the degradation of O-phosphonooxymethyl propofol is pH dependent. In particular, higher pH conditions generally result in lower rates of hydrolysis (degradation) of the prodrug, while lower pH conditions increase the rate of hydrolysis. At a pH of about 9-9.5, which is suitable for intravenous injection, the least amount of hydrolysis is observed at accelerated stability conditions of 40° C., 75% RH. Table II illustrates the formation of propofol (DIP) over a range of pH conditions.

TABLE II
DIP formation (% w/v) at Accelerated
Stability Conditions (40° C./75% RH)

| Time | pH 6.9 | pH 7.4 | pH 8.0 | pH 8.5 | pH 9.1 | pH 9.9 |
|---|---|---|---|---|---|---|
| 1 month | 1.22 | 0.83 | 0.32 | 0.15 | NMT | NMT |
| | | | | | LOQ | LOQ |
| | | | | | (0.05%) | (0.05%) |
| 3 months | 1.90 | 0.67 | 0.76 | 0.41 | 0.12 | NMT |
| | | | | | | LOQ |
| | | | | | | (0.05%) |
| 6 months | 3.47 | 2.14 | 0.72 | 0.73 | 0.22 | 0.09 |

NMT LOQ = not more than limit of quantitation

### Example 2

This example illustrates the color formation as a function of pH in O-phosphonooxymethyl propofol formulations containing an antioxidant, as described in WO 2003/057153 A2. Table III shows that a formulation prepared at pH 8.5 remained colorless after 6 months. Since even lower amounts of DIP are generated at pH>8.6, it was discovered that stable aqueous formulations can be prepared at pH>8.6 without requiring an antioxidant.

TABLE III
Impact of pH on Color Formation at Accelerated Stability Conditions

| Time | pH 6.9 | pH 7.4 | pH 8.0 | pH 8.5 | pH 9.1 | pH 9.9 |
|---|---|---|---|---|---|---|
| 1 month | Pale | Pale | Colorless | Colorless | Colorless | Colorless |
| | Yellow | Yellow | | | | |
| 3 months | Pale | Very | Very | Colorless | Pale | Colorless |
| | Yellow | pale | pale | | Yellow | |
| | | Yellow | Yellow | | | |
| 6 months | Very | Very | Very | Colorless | Colorless | Colorless |
| | pale | pale | pale | | | |
| | Yellow | Yellow | Yellow | | | |

### Example 3

This example illustrates the stability of an O-phosphonooxymethyl propofol (40 mg/mL) formulation prepared without antioxidant. The formulation contained 0.12% (w/v) 2-amino-2-hydroxymethyl-1,3-propanediol and 10 mmol sodium bicarbonate (pH 9.0-9.3). As illustrated in Table IV below, under accelerated stability conditions (40° C., 75% RH) the formulation remained colorless after 3 months storage and pH remained substantially constant.

TABLE IV
Stability of Antioxidant-free Formulation
at Accelerated Stability Conditions

| Time | Appearance | pH |
|---|---|---|
| 0 | Clear, Colorless | 9.31 |
| 1 weeks | Clear, Colorless | 9.27 |
| 2 weeks | Clear, Colorless | 9.27 |
| 4 weeks | Clear, Colorless | 9.31 |
| 5 weeks | Clear, Colorless | 9.34 |
| 6 weeks | Clear, Colorless | 9.34 |
| 8 weeks | Clear, Colorless | 9.39 |
| 12 weeks | Clear, Colorless | 9.29 |

### Example 4

This example illustrates pH stability over time of O-phosphonooxymethyl propofol formulations containing 10 mmol (0.12%) 2-amino-2-hydroxymethyl-1,3-propanediol (TRIS) and varying amounts of sodium bicarbonate. As indicated in Table V below, some of the formulations contained an antioxidant (MTG) while others did not.

TABLE V
pH Stability of Various Aqueous Formulations Over Time

| | pH as | Time | pH at | pH at | pH at |
|---|---|---|---|---|---|
| Formulation | prepared | 0 | 2 week | 4 week | 12 week |
| 40 mg/ml, 0.12% TRIS, | 8.6 | 8.61 | 8.74 | 8.96 | 9.08 |
| 10 mm Na bicarbonate | | | | | |
| 40 mg/ml, 0.25% MTG, | 8.6 | 8.62 | 8.7 | 8.78 | 8.83 |
| 0.12% TRIS, 10 mm Na | | | | | |
| bicarbonate | | | | | |
| 40 mg/ml, 0.12% TRIS, | 9 | 9.03 | 9.05 | 9.14 | 9.24 |
| 10 mm Na bicarbonate | | | | | |
| 40 mg/ml, 0.12% TRIS, | 9.28* | 9.28 | 9.27 | 9.34 | 9.35 |
| 10 mm Na bicarbonate | | | | | |
| 40 mg/ml, 0.25% MTG, | 8.92* | 8.92 | 9.09 | 9.09 | 9.05 |
| 0.12% TRIS, 10 mm Na | | | | | |
| bicarbonate | | | | | |
| 40 mg/ml, 0.12% TRIS, | 8.6 | 8.66 | 8.7 | 8.81 | 8.84 |
| 5 mm Na bicarbonate | | | | | |
| 40 mg/ml, 0.12% TRIS, | 9 | 8.99 | 9.02 | 9.09 | 9.13 |
| 5 mm Na bicarbonate | | | | | |
| 40 mg/ml, 0.12% TRIS, | 9.46* | 9.46 | 9.44 | 9.44 | 9.39 |
| 5 mm Na bicarbonate | | | | | |
| 40 mg/ml, 0.12% TRIS, | 8.6 | 8.66 | 8.7 | 8.72 | 8.66 |
| 2.5 mm Na bicarbonate | | | | | |
| 35 mg/ml, 0.25% MTG, | 8.6 | 8.87 | 8.62 | 8.51 | 8.16 |
| 0.12% TRIS | | | | | |

*pH was not adjusted

While particular embodiments of the present invention have been described and illustrated, it should be understood that the invention is not limited thereto since modifications may be made by persons skilled in the art. The present application contemplates any and all modifications that fall within the spirit and scope of the underlying invention as disclosed and claimed herein.

We claim:

1. A pharmaceutical formulation comprising in an aqueous medium a therapeutically effective amount of a compound represented by Formula I: 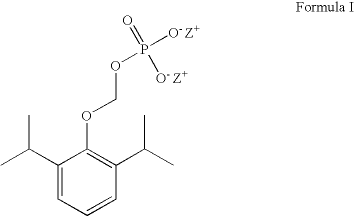
wherein each Z is independently selected from the group consisting of hydrogen, alkali metal ion, and amine; wherein the formulation is substantially free of antioxidant.

2. The formulation of claim 1 further comprising a buffer.

3. The formulation of claim 2 wherein the buffer comprises a combination of 2-amino-2-hydroxymethyl-1,3-propanediol and sodium bicarbonate.

4. The formulation of claim 2 which is buffered in a pH range of about 8 to about 12.

5. The formulation of claim 4 which is buffered in a pH range of about 9 to about 10.

6. A plastic container containing the formulation of claim 1.

7. The plastic container of claim 6 which is blow-filled vial.

8. A pharmaceutical formulation comprising in an aqueous medium:
(i) a therapeutically effective amount of O-phosphonooxymethyl propofol;
(ii) from about 2 to about 50 mmol 2-amino-2-hydroxymethyl-1,3-propanediol; and
(iii) from about 2 to about 50 mmol sodium bicarbonate;
wherein the formulation is buffered in a pH range of about 8 to about 12.

9. The formulation of claim 8 which is buffered in a pH range of about 9 to about 10.

10. The formulation of claim 8 which contains from about 2 to about 25 mmol 2-amino-2-hydroxymethyl-1,3-propanediol.

11. The formulation of claim 8 which contains from about 2 to about 25 mmol sodium bicarbonate.

12. A plastic container containing the formulation of claim 8.

13. The plastic container of claim 12 which is blow-filled vial.
